(19) United States
(12) Patent Application Publication
(10) Pub. No.: US 2006/0003965 A1
(43) Pub. Date: Jan. 5, 2006

(54) N-acetyl-d-glucosamine (nag) supplemented food products and beverages

(76) Inventors: Lawrence D. Fosdick, Oskaloosa, IA (US); John A. Bohlmann, Ottumwa, IA (US)

Correspondence Address: CARGILL, INCORPORATED, LAW DEPARTMENT, P. O. BOX 5624, MINNEAPOLIS, MN, 55440-5624, US

(21) Appl. No.: 10/533,414

(22) PCT Filed: Oct. 31, 2003
(86) PCT No.: PCT/US03/34846

§ 371 Date: Apr. 29, 2005

(30) Foreign Application Priority Data
Nov. 1, 2002 (US) 60423,119

(51) Int. Cl. A61K 31/7008; A61K 47/00

(52) U.S. Cl. 514/62

(57) ABSTRACT

Food products and beverages which include N-acetyl-D-glucosamine (NAG) are provided, as are methods of their use and preparation. Embodiments of the supplemented food products and beverages are heated to high temperatures, such as those used in pasteurization, without significant adverse effects on taste, color, odor and/or texture.

### CROSS REFERENCE TO RELATED APPLICATIONS

This claims the benefit of U.S. Provisional Application No. 60/423,119, filed Nov. 1, 2002, and is a continuation-in-part of PCT/US02/25121, filed Aug. 7, 2002, which claims priority from U.S. application Ser. No. 09/924,865, filed Aug. 8, 2001, each of which is incorporated herein by reference.

### FIELD

This application relates to food products and beverages that include N-acetyl-D-glucosamine (NAG), as well as methods of making and using NAG-supplemented food products and beverages.

### BACKGROUND

Food and beverage supplements can supply consumers with the necessary vitamins and minerals specified in the recommended daily allowances (RDA) provided by the U.S. government. Examples of such nutritionally-balanced snack bars are disclosed in U.S. Pat. Nos. 6,432,929; 6,391,864; 4,543,262; and 3,814,819. Examples of such nutritionally-balanced beverages are disclosed in U.S. Pat. Nos. 3,894,148; 4,309,417; 4,312,856; 4,322,407; 6,432,929; and 6,391,864 as well as EP Application No. EP 0 681 434.

Dietary cartilage supplements are effective in reducing the symptoms of osteoarthritis and joint pain. Examples of such cartilage supplements include glucosamine (GLCN) hydrochloride, GLCN sulfate, chondroitin sulfate, hyaluronic acid (which is comprised of a repeating disaccharide of N-acetyl-D-glucosamine and D-glucuronic acid), and cetyl myristoleate (CM). Two commonly used cartilage supplements are GLCN hydrochloride and GLCN sulfate.

It has been disclosed and the industry has followed the belief that exposure of GLCN to relatively high temperatures inactivated GLCN. In attempt to overcome this limitation, U.S. Pat. No. 6,423,929 teaches that beverages that include GLCN are prepared using a process that requires two separate heating steps to minimize chemical alteration of GLCN. A juice drink base (without GLCN) is prepared using pasteurization at about 195° F. for 42 seconds. A separate GLCN water-based solution is prepared at a temperature of below 160° F., such that the GLCN is not inactivated. The juice drink base and the GLCN solution are then mixed to form a GLCN-supplemented beverage. Processing a beverage using two different solutions at two different temperatures could be relatively expensive and difficult to implement.

### SUMMARY

Food products and beverages, for human or animal consumption, which include N-acetyl-D-glucosamine (NAG), and methods for making and using such food products and beverages, are disclosed herein. In particular examples, the disclosed food products and beverages supplemented with NAG are exposed to high temperatures without significant adverse effects on taste, color, odor, and/or texture of the NAG-supplemented food products and beverages. In alternative or additional examples, the NAG present in the disclosed food products and beverages does not significantly degrade when exposed to high temperature applications. For example, the amount of NAG present in a NAG food product or NAG beverage following exposure to a high temperature is at least about 70% of an amount of NAG present in the food or beverage prior to the exposure to high temperature. In some examples, the NAG-supplemented food products and beverages are consumed to treat or prevent disease, such as a cartilage dysfunction, a food allergy, or a skin disorder.

Because the nitrogen in NAG is in the neutral amide, the use of NAG over other dietary cartilage supplements, such as GLCN, which is an acidic sugar, certain embodiments of the disclosed food products and beverages provide various additional advantages. For example, in some examples, NAG has no substantial effect on the pH of the supplemented food product or beverage. In particular examples, NAG also imparts sweetness to the supplemented food or beverage, making it suitable as a replacement for some or the entire sweetener in the food or beverage. In some examples, NAG does not participate in Maillard chemistry, and therefore does not contribute undesirable color to the product.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. For example, reference to “a beverage” includes a plurality of such beverages and reference to “the food product” includes reference to one or more food products and equivalents thereof known to those skilled in the art, and so forth. Similarly, the word “or” is intended to include “and” unless the context clearly indicates otherwise. Hence “comprising A or B” means including A, or B, or A and B.

Unless otherwise indicated, all numbers expressing quantities of ingredients, temperatures, time periods, and so forth used in the specification and claims are to be understood as being modified by the term “about” whether explicitly stated or not. Accordingly, unless indicated clearly to the contrary, the numerical parameters set forth are approximations.

Unless explained otherwise, all technical and scientific terms used herein have the same meaning as commonly understood to one of ordinary skill in the art to which this disclosure belongs.

Administer: To cause a subject to receive something. As used herein, administration of the disclosed food products or beverages supplemented with NAG is oral, for example by ingestion.

Beverage: Any drink suitable for ingestion. Includes beverages in their liquid form, such as juice or soda, concentrates, as well as those in a dry or powered form, such as a tea, instant coffee, or hot chocolate mix.

Cartilage dysfunction: A disorder in a subject that results in joint pain or decreased joint mobility, for example arthritis, such as osteoarthritis.

Cartilage supplement: An agent that reduces joint pain, increases joint mobility, reduces swelling, or stimulates joint healing in a subject. In particular examples, it is an agent that delays or halts the onset of osteoarthritis. Examples include, but are not limited to: NAG, glucosamine, chondroitin sulfate, hyaluronic acid, chitin, cetyl myristoleate, essential fatty acids, MSM, SAMe, oligoglucosamine, and oligomers of NAG.

Comprises: A term that means including.

High Temperature: As used herein, refers to temperatures typically used in heat pasteurization to significantly reduce the presence of undesirable microorganisms, or in other thermal processing methods, such as baking, broiling, boiling, roasting, or frying. A NAG food product or NAG beverage can be exposed to a high temperature for an amount of time as needed to achieve a desired effect, for example to destroy objectionable microorganisms, to cook a product, or to bring a NAG product or NAG beverage to a boil.

Particular, non-limiting examples of high temperatures include, but are not limited to temperatures of at least about 160° F. (about 71° C.), such as temperatures of at least about 161° F. (about 71.5° C.), at least about 180° F. (82° C.), at least about 194° F. (about 90° C.), at least about 200° F. (about 94° C.), at least about 212° F. (about 100° C.), at least about 220° F. (about 104° C.), at least about 280° F. (about 138° C.), at least about 300° F. (about 149° C.), at least about 325° F. (about 163° C.), at least about 350° F. (about 177° C.), at least about 375° F. (about 191° C.), at least about 400° F. (about 204° C.), at least about 450° F. (about 232° C.), or at least about 500° F. (about 260° C.).

In particular examples, high temperatures include temperatures in the range of about 160° F. to about 500° F., such at about 160° F. to about 350° F., about 160° F. to about 212° F., about 160° F. to about 200° F., or about 194° F. to about 212° F.

N-acetyl-D-glucosamine (NAG): As used herein, refers to monomers of NAG, as well as oligomers of NAG that have the same or similar thermal tolerance as disclosed herein. For example, NAG and NAG oligomers can be introduced into a food or beverage, and be subsequently subjected or exposed to a high temperature, without a resulting significant adverse effect on the taste, color, odor, or texture of the food or beverage supplemented with NAG.

Oligomers of NAG are those having a degree of polymerization, such as a polymer of 2-6 NAG molecules. Examples of NAG oligomers include, but are not limited to: dimers, trimers, tetramers, pentamers, and hexamers of NAG, which have the same or similar thermal tolerance as disclosed herein.

NAG can be obtained from any suitable source. In certain examples, NAG is a NAG composition that is derived from shellfish, cartilage, bacteria, and/or fungal biomass.

In one example, NAG is derived from fungal biomass containing chitin (for example see PCT Publication WO 03/013435). A fungal biomass that contains chitin and glucan is typically degraded to produce NAG. The chitin and glucan can be degraded enzymatically (such as using enzymes secreted by eukaryotic or prokaryotic microorganisms, for example chitinases, glucanases, and β-N-acetyl-gluosaminidases) or chemically. When enzymes are used, the degradation reaction can be maintained at a pH of from about 4.0 to about 6.0 at about 20° C. to about 45° C.

Suitable starting materials include microbial fungal sources, such as fungal sources derived from Aspergillus sp., Penicillium sp., Mucor sp., and combinations thereof. When NAG is derived from fungal biomass, it will not pose a hazard to persons who have shellfish allergies because tropomyosin and other such muscle-derived proteins are not present in fungal biomass. Therefore, food products and beverages containing NAG derived from fungal biomass will be tolerated by persons who have shellfish allergies. In addition, because NAG derived from fungal biomass is not derived from shellfish (or any animal source), such NAG-containing food products and beverages are qualified for kosher status and may be consumed by strict vegetarians.

In another example, NAG is derived from a bacterial source (for example U.S. Patent Application No. 2002/0160459). In one embodiment, bacteria, such as E. coli, are transformed with a recombinant nucleic acid encoding N-glucosamine-6-phosphate synthase, allowing the bacteria to produce the recombinant protein, then recovering NAG from the fermentation medium.

NAG beverage: A beverage that contains NAG, for example at least about 1 mg NAG per serving, such as at least about 100 mg, at least about 1 g, or at least about 5 g NAG per serving. In particular examples, the amount of NAG in a beverage is about 250 mg to about 750 mg per serving. A heat pasteurized NAG beverage is one that includes NAG in the beverage when the beverage is exposed to high temperatures used in heat pasteurization.

NAG food product: A food product that contains NAG, for example at least about 1 mg NAG per serving, at least about 100 mg, at least about 1 g, or at least about 5 g NAG per serving. In particular examples, the amount of NAG in a food product is about 250 mg to about 750 mg per serving. A pasteurized NAG food product is one that includes NAG in the food product when the food product is exposed to high temperatures, such as those used in baking.

Non-acidified food product: A food product that does not contain organic acids such as citric, acetic, or fumeric.

Pasteurize: A method used to significantly reduce the presence of objectionable organisms (such as bacteria) in a NAG food product or NAG beverage by exposing the food or beverage to heat or irradiation for a period of time. Exemplary methods of pasteurization include thermal processing at a high temperature (referred to herein as heat pasteurization), filtration, such as microfiltration, and irradiation processing (such as heating in a microwave or its industrial equivalent). Ideally, pasteurization does not substantially chemically alter a NAG food product or NAG beverage, and does not substantially affect the taste or mouthfeel of the NAG food product or NAG beverage. As used herein, “heat pasteurization” or “heat pasteurized” does not include pasteurization by filtration, or irradiation.

In particular examples, heat pasteurization reduces the number of colony forming units (cfus) present in a NAG food product or NAG beverage prior to heat pasteurization by at least 50%, such as at least 70%, at least 75%, at least 80%, at least 90%, at least 95%, or even at least 98%. Heat pasteurized products can be subsequently cooled quickly to about 38° F. to retard the growth of surviving organisms.

Particular, non-limiting examples of heat pasteurization temperatures include temperatures of at least about 160° F. (about 71° C.), such as temperatures of at least about 161° F. (about 71.5° C.), at least about 180° F. (82° C.), at least 194° F. (about 90° C.), at least about 200° F. (about 94° C.), at least about 212° F. (about 100° C.), at least about 220° F. (about 104° C.), at least about 280° F. (about 138° C.), or at least about 300° F. (about 149° C.). In particular examples, heat pasteurization temperatures include temperatures in the range of about 161° F. to about 300° F., such at about 161° F. to about 220° F., about 161° F. to about 212° F., about 161° F. to about 200° F., 165° F. to about 220° F., about 165° F. to about 212° F., about 176° F. to about 220° F., or about 176° F. to about 212° F.

Particular examples of heat pasteurization temperatures and incubation times include, but are not limited to: at least about 15 seconds at a temperature of at least about 160° F., at least about 30 seconds at a temperature of at least about 161° F., or at least about 20 minutes at about 350° F. Other examples include, but are not limited to: about 161° F. for 15 seconds, about 195° F. for about 42 seconds (such as about 195±4° F. for about 42±4 seconds), about 200° F. for less than 40 seconds (such as about 200±5° F. for about 40±5 seconds), about 165° F. for about 3 minutes (such as about 165±5° F. for about 180±10 seconds), and at or above 280° F. for about 1-2 seconds (for example to ultrapasteurize milk). If ultrapasteurization is desired, pasteurization temperatures can be increased to about 280° F. or greater (such as about 300° F.), with incubation for a shorter period of time, such as 1-2 seconds.

Preventing disease: A therapeutic intervention that inhibits the full development of a disease, for example preventing development of osteoarthritis in a subject having cartilage dysfunction.

Serving: A serving is the amount of food or beverage a person or animal would customarily eat in one time. The serving size can often times be found on the Nutrition Facts label on the food or beverage. Serving sizes are also shown on the USDA Food Pyramid. For bulk products, such as breakfast cereal and flour, a serving is usually represented in common household terms, such as cup, tablespoon, teaspoon, or fluid ounce. For products that come in discrete units, such as bread and cookies, a serving size is usually listed as the number of units that constitute a serving, such as three cookies or two slices of bread.

Shellfish: A term for mollusks and crustaceans used as food. Exemplary shellfish include clams, snails, mussels, oysters, scallops, shrimp, lobster, and crayfish. Components of the shell or exoskeleton of these organisms can be converted into GLCN using known techniques.

Shellfish protein: A protein present in a shellfish, such as those that are allergenic in humans having shellfish allergies. Exemplary shellfish proteins include, but are not limited to, shellfish muscle proteins, such as tropomyosin.

Skin disorder: A disease or disorder in a subject that negatively affects the skin, and benefits from collagen formation. Examples include, but are not limited to: a wound, wrinkles, and acne. When NAG is used to treat a skin disorder, NAG can be introduced into products used on the skin, such as topical lotions and creams. Alternatively or in addition, NAG can be introduced into food products and beverages and consumed by a subject in need of treatment or prevention of a skin disorder.

Subject: Living multicellular vertebrate organisms, a category which includes both human and veterinary subjects, for example, mammals, rodents, and birds.

Therapeutically Effective Amount: An amount sufficient to achieve a desired biological effect. In one example, it is an amount that is effective to alleviate or reduce symptoms associated with cartilage dysfunction, such as pain, swelling, and decreased mobility, by more than a desired amount. In another example, it is an amount that is effective to stabilize symptoms associated with cartilage dysfunction, such that the symptoms do not worsen. In particular examples, it is a concentration of NAG that is effective to alleviate, reduce, or stabilize symptoms associated with cartilage dysfunction, alone or in combination with other agent, such as in a subject to whom NAG is administered.

In one example, it is an amount that is effective to alleviate or reduce symptoms associated with a skin disorder, such as promoting the healing of a wound or reducing the appearance of wrinkles, by more than a desired amount. In another example, it is an amount that is effective to stabilize symptoms associated with a skin disorder, such that the symptoms do not worsen. In particular examples, it is a concentration of NAG that is effective to alleviate, reduce, or stabilize symptoms associated with a skin disorder, alone or in combination with other agent, such as in a subject to whom NAG is administered.

In one example, a therapeutically effective amount also includes a quantity of NAG sufficient to achieve a desired effect in a subject being treated. For instance, it can be an amount necessary to improve signs or symptoms a disease, such as osteoarthritis, a skin disorder, or a wound.

The NAG-containing food products and beverages disclosed herein have equal application in medical and veterinary settings. Therefore, the general term “subject being treated” is understood to include all animals (such as humans, apes, dogs, cats, horses, and cows) that require treatment of a cartilage dysfunction or skin disorder, such as a wound.

Thermal tolerance: Refers to the ability of NAG to be exposed to a high temperature, without a resulting significant adverse effect on the taste, color, odor, or texture of a food or beverage supplemented with NAG, when NAG is present in the food or beverage during exposure to a high temperature. In particular examples, the amount of NAG present in a NAG food product or NAG beverage following exposure to a high temperature, demonstrating that NAG is thermally tolerant, is at least 70% of the original amount of NAG present, for example at least about 75%, at least about 77%, at least about 80%, at least about 90%, at least about 95%, at least about 98%, at least about 99%, or even 100% (no loss of NAG).

In contrast, food and beverage products including GLCN exposed to high temperatures oftentimes have undesirable characteristics, such as an unpleasant taste and undesirable browning, when GLCN is present in the food or beverage during exposure to a high temperature.

Treat: To alleviate or reduce one or more of the symptoms of a disorder, such as a cartilage dysfunction, wound or skin disorder, or to stabilize such a condition.

### Advantages of NAG Over Other Cartilage Supplements

Certain embodiments of the disclosed food products and beverages including NAG as a dietary cartilage supplement offer one or more advantages over the use of GLCN. In one example, the disclosed food products and beverages containing NAG can be exposed to high temperatures, such as those used in heat pasteurization or baking, without a significant adverse affect on the resulting taste, color, odor, or texture of the food or beverage. In certain examples, NAG food products and NAG beverages are more stable at neutral or high pH values than are similar products that include GLCN.

The nitrogen in NAG is in the neutral amide form, and therefore in some examples has no substantial effect on the pH of the supplemented food product or beverage. This pH tolerance makes NAG suitable for a wide range of foods and beverages, such as dairy products which can curdle at lower pH. In addition, because NAG is not a salt, in certain embodiments subjects needing to lower their salt intake would not be adversely affected by consuming NAG-supplemented beverages or food products. Because NAG can impart sweetness to a supplemented food or beverage, in some examples it is used as a replacement for some or the entire sweetener in the food or beverage. For example, NAG can replace at least about 1% of a sweetener in a NAG food or NAG beverage, such as at least about 5% or at least about 10%. In certain embodiments, NAG does not participate in Maillard chemistry, and so does not contribute undesirable color to the product. Maillard chemistry is the nonenzymatic browning observed when amine-containing species, such as amino acids or proteins, react with carbohydrates during heating. Maillard chemistry contributes to the browning obtained in many baked goods, such as bread and cookies.

### Food and Beverages Supplemented with NAG

Disclosed herein are beverages and food products supplemented with NAG, and that are exposed to high temperatures, such as during processing or preparation of the food or beverage. For example, NAG-supplemented food products and beverages can be heat pasteurized or baked, without significant loss of NAG. For example, following exposure of NAG-supplemented food products and beverages to high temperatures, the amount of NAG remaining in the food product or beverage is at least 70%, such as at least 90%.

The amount of NAG added to the beverage or food product will depend on the desired concentration. In certain examples, NAG is present in the disclosed food products and beverages in amounts effective for promoting the development of connective tissue in the body, alone or in combination with other agents, such as cartilage promoting agents. In some embodiments, daily NAG dosages include at least about 250 mg, at least about 500 mg, at least about 1000 mg, at least about 2000 mg, or even about at least 3000 mg. Particular NAG dosage ranges include, but are not limited to, a range of about 500 mg to about 3000 mg, such as about 1000 mg to about 2000 mg, such as about 1500 mg of NAG.

Certain embodiments of the disclosed amounts of NAG that can be included in a food or beverage include, at least about 0.001 g NAG/serving, such as at least 0.01 g NAG/serving, at least about 0.05 g NAG/serving, at least about 0.1 g NAG/serving, at least about 0.25 g NAG/serving, at least about 0.5 g NAG/serving, at least about 0.75 g NAG/serving, at least about 1 g NAG/serving, at least about 1.5 g NAG/serving, at least about 3.0 g NAG/serving, at least about 5 g NAG/serving, at least about 10 g NAG/serving, or at least about 20 g NAG/serving. In other examples, the amount of NAG added is about 1 g NAG/1000 g of product to about 1 g NAG/0.1 g of product, such as about 1 g NAG/10 g product to about 1 g NAG/0.5 g product.

Certain embodiments of the disclosed NAG-supplemented food products and beverages also include one or more other cartilage supplements, vitamins, minerals, fats, proteins, carbohydrates, sweeteners, organic acids, glucose or combinations thereof. In addition, other agents that treat cartilage dysfunction or skin disorders can also be included in the disclosed NAG-supplemented food products and beverages.

In some examples, the NAG food products and NAG beverages do not contain detectable amounts of shellfish proteins, such as those muscle proteins which are allergenic in some humans (that is, those persons having shellfish allergies). In some examples, in order to decrease or even eliminate the presence of shellfish proteins, NAG is derived from fungal biomass, bacteria, or cartilage, instead of from shellfish.

In certain embodiments, NAG-supplemented beverages (or food products) can be heat pasteurized at a high temperature, wherein NAG is present in the beverage (or food product) during the pasteurization. Particular non-limiting heat pasteurization temperatures include, at least about 160° F., at least about 180° F., at least about 200° F., at least about 250° F., or at least about 300° F. Heat pasteurization in particular embodiments can also include exposure to high pressure, such as about 121° C. at 1 atm for 15 minutes. In one example, NAG is included in a coffee, tea, or cocoa mixture (such as a pre-prepared packet) to which boiling or heated water (or other liquid such as milk) is added.

NAG beverages, such as those including at least about 0.01 g NAG per serving, at a temperature of at least about 160° F., such as at least about 161° F., such as at least about 165° F., at least about 194° F., at least about 200° F., at least about 212° F., at least about 220° F., or even at least about 280° F., are encompassed by this disclosure.

Non-limiting examples of beverages that can be supplemented with NAG include naturally or artificially flavored fruit or vegetable juices such as apple juice, carrot juice, cherry juice, cranberry juice, grape juice, grapefruit juice, orange juice, pear juice, tomato juice, or a combination thereof; milk; commercially available sports drinks (sugar or juice based) such as Gatorade®, Powerade®, and Allsport®; soda; Tang®; flavored waters; soy milk; and commercially available nutritionally-balanced beverages such as Ensure® beverage. The beverage can be carbonated or non-carbonated. In particular embodiments, the beverage is in a concentrated form for later dilution by the consumer or ready-to-drink. Alcoholic beverages are also encompassed by this disclosure, such as wine, wine coolers, malt beverages and coolers, and beer.

Certain embodiments of the disclosed food products are thermally processed after NAG is included in the food composition. Examples of thermal processing include, but are not limited to, baking, roasting, broiling, and frying. Non-limiting examples of food products include flour- and grain-based products, such as bakery products, for example bread, cookies, muffins, rolls, brownies, pies and cakes (or mixes to prepare such products, such as cake mixes). Other non-limiting examples include breakfast cereals, nutrition bars, snack bars, granola bars, animal feed products, bread crumbs, yogurt, gum, candy, and canned-goods, such as soups, pastas, vegetables, fruits (such as pie fillings) and meats.

In particular examples, NAG food products are exposed to a high temperature, such as a temperature used in baking or frying, such as at least about 300° F., at least about 325° F., at least about 350° F., at least about 375° F., at least about 400° F., at least about 425° F., at least about 450° F., and even such as at least about 500° F.

NAG food products, such as those including at least about 0.01 g NAG per serving and no detectable shellfish proteins, at a temperature of at least about 160° F., such as at least about 161° F., such as at least about 165° F., at least about 194° F., at least about 200° F., at least about 212° F., at least about 300° F., at least about 350° F., at least about 400° F., or even at least about 500° F., are encompassed by this disclosure.

### Methods of Preparing Foods and Beverages Supplemented with NAG

Methods of preparing beverages that include NAG are disclosed. In one example, the method includes heat pasteurizing the beverage, wherein NAG is present in the beverage during pasteurization. In another example, the method includes combining at least about 0.01 g NAG per serving and a beverage, thereby forming a NAG-beverage, and then exposing the NAG-beverage to heat pasteurization at a temperature of at least 160° F.

Also disclosed are methods for preparing NAG-supplemented food products. In one example, the method includes combining a food product and at least about 0.01 g NAG per serving, wherein NAG is derived from fungal biomass, bacteria, or cartilage such that no detectable shellfish allergens are present, and then exposing the NAG food product to a high temperature, such as at least 160° F. For example, the NAG food product can be baked; broiled; boiled; sterilized; canned; roasted; fried; such as by heating in an oven, autoclave, microwave oven (or their industrial equivalents).

### Treatment Using NAG

A method of treating a cartilage dysfunction in a subject by administering the disclosed NAG-supplemented food products or beverages, is disclosed. In some examples, treatment alleviates or reduces the symptoms of cartilage dysfunction, such as increases joint mobility, reduces pain or reduces swelling in the subject. In some examples, treatment stabilizes the symptoms of cartilage dysfunction, such that the cartilage dysfunction is not exacerbated. Examples of cartilage dysfunction include, but are not limited to, joint pain and osteoarthritis.

Also disclosed are methods of treating a sldn disorder in a subject by administering the disclosed NAG-supplemented food products or beverages to a subject. In some examples, treatment alleviates or reduces the symptoms of a skin disorder, such as promotes wound healing in the subject. In some examples, treatment stabilizes the symptoms of a skin disorder, such that the skin disorder is not exacerbated. Examples of skin disorders include, but are not limited to, wounds and wrinkles.

A method for treating food allergies in a subject by administering the disclosed NAG-supplemented food products or beverages to the subject is disclosed. In some examples, treatment alleviates or reduces the symptoms of a food allergy, such as reduces the inflammatory response to the food in the subject. In some examples, treatment stabilizes the symptoms of a food allergy, such that the food allergy is not exacerbated.

The subject treated can be a human or veterinary subject suffering from cartilage dysfunction, skin disorder or food allergy (for example see WO 93/14766A1). An effective amount of NAG can be administered in a single serving, or in several servings, for example daily, during a course of treatment. However, the effective amount can depend on the subject being treated, the severity and type of the condition being treated, and the manner of administration (food product versus beverage). A typical amount of GLCN or NAG delivered in dietary supplement products is about 1.5 g/day, in a single or in multiple administrations. For example, if the subject was to receive multiple administrations in a single day, the subject might receive three servings of NAG, each containing about 0.5 g NAG. In certain embodiments, NAG is administered at about at least about 0.01 g NAG/day, about at least 0.05 g NAG/day, about at least 0.1 g NAG/day, about at least 0.25 g NAG/day, about at least 0.5 g NAG/day, about at least 0.75 g NAG/day, about at least 1.0 g NAG/day, about at least 1.5 g NAG/day, about at least 3.0 g NAG/day, about at least 5.0 g NAG/day, about at least 10.0 g NAG/day, or even about at least 20.0 g NAG/day.

### EXAMPLE 1

### Rice Krispies® Treats

Rice Krispies® Treats were used as the basis for incorporation of samples. According to the manufacturer, two treats are a serving. To prepare the samples, four batches of Rice Krispies® Treats were prepared for sensory testing as follows:
batch 1. Control (no GLCN, no NAG).
batch 2. GLCN (glucosamine hydrochloride derived from fungal biomass, Cargill, Minneapolis, Minn., Lot No. GP015-A).
batch 3. GLCN (glucosamine hydrochloride derived from shellfish, Batch 011107, Index # 9152, V.L. Clark Chemical Co., Inc., Union, Mo.).
batch 4. NAG (N-Acetyl-D-glucosamine, Lot # 27294A, Catalog # A-102-2, Ferro Pfanstiehl, Waukegan, Ill.).

According to the manufacturer's instructions, the recipe prepares 24 treats, so 4 batches were prepared from the recipe. Each batch consisted of six treats, or three servings. For servings which included GLCN or NAG, the serving (2 treats) had 0.75 g GLCN or NAG, or 0.375 g per treat added, which reflects a typical amount of GLCN or NAG delivered in dietary supplement products (0.25 g-1.5 g/serving). Therefore, except for the control batch, each batch had 2.25 g of GLCN or NAG added (0.375 g GLCN or NAG per treat×6 treats in batch=2.25 g GLCN or NAG per batch). GLCN or NAG was added to the melted marshmallow (melt marshmallow in a 1000 watt Amana Radarange microwave following manufacturer's instructions) prior to adding the Rice Krispies®, to evenly disperse the minor dry ingredients. Here, the samples were not heated to high temperatures.

Samples were tested within one day of preparing. Samples were tested blindly against a marked control, and a blind control was included. Panelists were asked to compare each sample to the control and comment. Panelists received the following instructions:
1. Taste the control sample first.
2. Compare all other samples to the control and write descriptors in the table (some potential comments might be whether or not the sample is hard, soft, brittle, crumbly, chewy, or grainy).

3. Numerically rate whether the samples are better or worse than the control, using this scale:

| | | | | | | | | | | |
|---|---|---|---|---|---|---|---|---|---|---|
| −5 | −4 | −3 | −2 | −1 | 0 | +1 | +2 | +3 | +4 | +5 |

| | | |
|---|---|---|
| Worse than control | Same as control | Better than control |

4. Please do not discuss the results with any other panelists until all sheets are turned in.

The 12 Panelists' results are shown in Table 1.

TABLE 1
Results of Rice Krispies ® Treats.*

| | | | Mouthfeel/ | | |
|---|---|---|---|---|---|
| Sample | Color | Odor | texture | Taste | Comments (other than “same”) |
| Control | 0 | −0.3 | −0.4 | 0 | A little crunchier (3) |
| NAG | 0 | +0.3 | +0.3 | +0.3 | Much soggier, not good, shinier, chewier, sweeter |
| | | | | | (2) |
| Fungal | 0 | −0.3 | −0.1 | −0.4 | A little crunchier, softer (2), not good, slight salt |
| GLCN | | | | | aftertaste, sour (3), slight aftertaste, sweeter, |
| | | | | | chewier, aftertaste, notice strange taste |
| Shellfish | 0 | 0 | 0 | −1.4 | Sour (3), softer (2), not good, chewier (3), tangy, |
| GLCN | | | | | slight aftertaste, sweeter, similar to aftertaste of |
| | | | | | cinnamon (burning), different but not unpleasant, |
| | | | | | aftertaste, stronger flavor |

*Results shown are the average rounded to nearest tenth.

As shown in Table 1, the results were validated by identification of the control, based on the control score of near “0” in all categories. In addition, Table 1 demonstrates that NAG was preferred by the panelists in Rice Krispies® Treats over either GLCN sample, and it was unexpectedly favored over the control. These results demonstrate that NAG has an advantage over GLCN when used in a high temperature food application, as NAG does not appear to adversely affect taste after being exposed to heated marshmallow.

### EXAMPLE 2

### Betty Crocker® Sugar Cookie Mix

A packet of Betty Crocker® dry cookie mix was used as the basis for incorporation of samples. A sugar cookie mix was used due to its light color and mild taste. According to the manufacturer, two cookies are one serving.

On package of sugar cookie mix prepares 36 cookies, so each batch consisted of nine cookies, or 4.5 servings. For servings which included GLCN or NAG, the serving (2 cookies) had 0.75 g GLCN or NAG, or 0.375 g per cookie added, which reflects a typical amount of GLCN or NAG delivered in dietary supplement products. Except for the control, to each batch 3.375 g of NAG or GLCN was added (0.375 g GLCN or NAG/cookie×9 cookies in batch=3.375 g GLCN or NAG/batch). Each batch was baked separately, so that the cookies were baked in the same places on the cookie sheet. GLCN or NAG was added to the dry mix and mixed with a spoon. Before baking each batch, 1/12 cup of oil, followed by ¼ of a beaten egg, was added. The mixture was stirred by spoon, then by hand, to form a soft dough. The dough was weighed and divided into nine even balls and placed at least two inches apart on an ungreased cookie sheet, then flattened in a crisscross pattern with a fork. Cookies were baked at 375° F. for 8-10 minutes on a shiny metal pan. Cookies were cooled for two minutes before removing from cookie sheet, then stored in airtight containers.

Samples were tested according to the methodology and instructions in Example 1. The 12 Panelists' results are shown in Table 2.

TABLE 2
Results of Betty Crocker ® Sugar Cookies.*

| | | | Mouthfeel/ | | |
|---|---|---|---|---|---|
| Sample | Color | Odor | texture | Taste | Comments (other than “same”) |
| Control | −0.3 | −0.2 | 0 | −0.1 | Darker around edges, harder |
| NAG | 0 | +0.1 | +0.2 | −0.1 | Slightly sweeter, more moist, chewier (3), |
| | | | | | looks lighter than control, crumbly, smells |
| | | | | | sweeter |
| Fungal | −1.5 | −0.3 | −0.7 | −1.7 | Crumbly (2), burnt sugar/caramel taste (4), |
| GLCN | | | | | darker and/or dark specs on bottom (10), |
| | | | | | slight cinnamon-like hot taste, strange |
| | | | | | aftertaste, taste & smell sweet, |
| | | | | | crunchier/harder (4), toasted odor, off smell, |
| | | | | | grey/brown border on bottom is unappealing |
| | | | | | (2), off/bitter aftertaste-almost salty |
| Shellfish | −1.0 | −0.1 | −0.2 | −0.8 | Crumbly (2), burnt taste, chewier, dark specs |
| GLCN | | | | | throughout and on bottom (8), smell sweet, |
| | | | | | taste sweet (2), chewy, crunchier, toasted |
| | | | | | odor, flour taste, color change on border is |
| | | | | | unappealing, firmer |

*Results are the average rounded to nearest tenth.

The results shown in Table 2 were validated by identification of the control, based on the control score of near “0” in all categories. As shown in Table 2, NAG was preferred by the panelists in sugar cookies over either GLCN sample, and the scores indicate it was even favored over the control. Only the GLCN samples showed significant darkening, indicating adverse interactions between the food components and GLCN during heating. Therefore, panelists did not like the taste or appearance of the GLCN samples, and the NAG sample was accepted or favored. These results demonstrate that NAG has an advantage over GLCN when used in a high temperature food application, as NAG does not appear to adversely affect taste or browning after being heated to 350° F., unlike GLCN. Because NAG enhanced the sweetness of the sugar cookies, NAG can be used to replace all or some of the sweetener, such as at least 5%, at least 10%, or at least 20% of a sweetener used in a beverage or food product.

### EXAMPLE 3

### Allsport® Sports Drink

Allsport® sports drink (The Monarch Company, Atlanta, Ga.) was used as the basis for incorporation of samples. Citrus Slam flavor was chosen because of its light color, which may help panelists observe any color change. According to the manufacturer, a 20-ounce bottle of Allsport® contains 2.5 servings.

For servings that included GLCN or NAG, the serving had 0.75 g GLCN or NAG added, which reflects a typical amount of GLCN or NAG delivered in dietary supplement products. Therefore, 1.875 g (0.75 g/serving GLCN or NAG×2.5 servings/bottle=1.875 g GLCN or NAG/bottle) of GLCN or NAG was added to a 20-ounce bottle of Allsport®. After the bottles were prepared, the contents were heated to 195±4° F. for 42±4 seconds to simulate a pasteurization step. Samples were heated in a 1000 watt Amana Radarange microwave oven in foam cups for the same amount of time. For a room temperature beverage, 150 seconds of heating time was necessary, and for a refrigerated beverage, 180 seconds was required. The beverages were cooled in their original bottles, ¾ submerged in an ice water bath, and hand rotated to simulate flash cooling.

Samples were tested according to the methodology and instructions in Example 1. The 12 Panelists' results are shown in Table 3.

TABLE 3
Results of Allsport ® sports drink.*

| | | | Mouthfeel/ | | |
|---|---|---|---|---|---|
| Sample | Color | Odor | texture | Taste | Comments (other than “same”) |
| Control | +0.1 | 0 | +0.4 | −0.1 | Less sour than others, sweeter (2), more of a |
| | | | | | carbonated feel, stronger odor (2), more |
| | | | | | tangerine taste, mouthfeel thicker |
| NAG | −0.1 | +0.1 | 0 | −0.2 | Less tangy/sour than others (3), sweeter, liked |
| | | | | | it better, crystals on top |
| Fungal | 0 | −0.3 | +0.1 | −0.3 | After/offtaste (2), “vitamin” like taste, |
| GLCN | | | | | sweeter, less odor, sour, yucky |
| Shellfish | −0.1 | −0.3 | −0.2 | −0.7 | Sweeter (2), sour, crystals on top, less odor, |
| GLCN | | | | | mouthfeel thicker, less tangy, flat and yucky |

*Results are the average rounded to nearest tenth.

As shown in Table 3, it was difficult for the panelists to ascertain differences between the samples. The control was not clearly differentiated from the NAG or fungal GLCN. However, the NAG samples were favored over both GLCN samples. These findings further demonstrate that taste and other factors are not adversely affected when NAG is used in a high temperature beverage application, such as those that require pasteurization. Further distinctions between NAG and GLCN may be observed if higher temperatures or extended times are used.

### EXAMPLE 4

### Nestle Toll House® Milk Chocolate Morsels

Nestle Toll House® Milk Chocolate Morsels were used as the basis for incorporation of samples. One serving of morsels is 14 g.

The shellfish GLCN was more granular than the other samples, so it was milled to disperse better in the chocolate. For servings which included GLCN or NAG, the serving (14 g) had 0.75 g GLCN or NAG added, which reflects a typical amount of GLCN or NAG delivered in dietary supplement products. As 217 g of morsels were used for the batches which had GLCN or NAG, 11.625 g of GLCN or NAG was added (217 g is 15.5 servings, 15.5 servings×0.75 g/serving=11.625 g). Each batch was heated in a 1000 watt Amana Radarange at medium-high power for one minute. The sample was stirred and again heated in the microwave for additional 20 second intervals until the chocolate was smooth. The temperature of the chocolate was recorded with a candy thermometer, and the GLCN or NAG added to the appropriate batches and stirred in quickly and thoroughly. Plastic spoons were used to drop teaspoon-sized amounts of chocolate onto wax paper, and were allowed to cool.

Samples were tested according to the methodology and instructions in Example 1. The 12 Panelists' results are shown in Table 4.

TABLE 4
Nestle Toll House ® Milk Chocolate Morsels results.*

| | | | Mouthfeel/ | | Comments (other |
|---|---|---|---|---|---|
| Sample | Color | Odor | texture | Taste | than “same”) |
| Control | 0 | 0 | 0 | +0.2 | Dry, rough, creamy |
| NAG | −0.8 | −0.3 | −1.4 | −0.9 | Dry/crumbly (4), rough |
| | | | | | surface (4), sticky in |
| | | | | | mouth, crunchy/grainy |
| | | | | | (2), flavor better than |
| | | | | | control (3), “thicker” |
| | | | | | taste not good, |
| | | | | | less sweet, off |
| | | | | | flavor, bitter |
| Fungal | 0 | −0.1 | −0.8 | −2.0 | Bitter (7), sour (2), |
| GLCN | | | | | grainy (4), salty (2), |
| | | | | | burnt taste, metallic, |
| | | | | | strange aftertaste |
| Shellfish | 0 | −0.1 | −2.8 | −2.5 | Crunchy/gritty (8), |
| GLCN | | | | | sour (2), bitter (4), |
| | | | | | salty (2), bad aftertaste |

*Results are the average rounded to nearest tenth.

As shown in Table 4, the results were validated; based on the control score of near “0” in all categories, the control was identified. The taste of NAG was preferred by the panelists in milk chocolate over either GLCN sample, and three panelists favored it over the control. Both GLCN samples were bitter or sour and grainy. The appearance of the NAG sample was less desirable, likely because when the NAG was added to the chocolate, it thickened the texture, making it more like fudge. These results demonstrate that NAG has an advantage over GLCN when used in a high temperature food application, as NAG does not appear to adversely affect taste after being heated, unlike GLCN.

### EXAMPLE 5

### Libby's White Grape Juicy Juice®

Libby's White Grape Juicy Juice® was used as the basis for incorporation of samples. White Grape was chosen due to its light color, to help panelists observe any color change. According to the manufacturer, a serving is 8 ounces.

For servings that included GLCN or NAG, the 8 ounce serving had 0.75 g GLCN or NAG added, which reflects a typical amount of GLCN or NAG delivered in dietary supplement products. A 64 ounce bottle of juice was split into four 16-ounce portions. To the appropriate batch, 1.5 g of GLCN or NAG (16 ounces is two servings, and 2 servings×0.75 g/serving=1.5 g) was added. After preparing the samples, they were heated to 195±4° F. to simulate a pasteurization step in a microwave, then cooled, as described in Example 3.

Samples were tested according to the methodology and instructions in Example 1. The 12 Panelists' results are shown in Table 5.

TABLE 5
Libby's White Grape Juicy Juice ® results.*

| | | | Mouthfeel/ | | Comments (other |
|---|---|---|---|---|---|
| Sample | Color | Odor | texture | Taste | than “same”) |
| Control | +0.1 | 0 | 0 | 0 | Slight off taste, more |
| | | | | | lingering taste |
| NAG | −0.3 | 0 | −0.1 | −0.5 | Sweeter, less sweet (2), |
| | | | | | rouge color, bitter (2), |
| | | | | | darker (3), lingering taste |
| Fungal | −0.2 | −0.3 | −0.3 | −0.6 | Darker (2), thicker |
| GLCN | | | | | mouthfeel (2), sweeter |
| | | | | | (2), stale/weaker (2), |
| | | | | | smooth, soapy, lasting |
| | | | | | aftertaste, tarty, odor |
| | | | | | masked |
| Shellfish | −0.4 | −0.1 | −0.3 | −1.1 | Darker (3), thicker |
| GLCN | | | | | mouthfeel, sweeter (2), |
| | | | | | soapy (2), tarty, bitter, |
| | | | | | less sweet, less fruity (2) |

*Results are the average rounded to nearest tenth.

The results shown in Table 5 were validated by identification of the control, based on the control score of near “0” in all categories. As shown in Table 5, it was difficult for the panelists to ascertain differences between the samples. However, NAG was preferred over the GLCN samples, but not overwhelmingly so. These results demonstrate that taste and other factors are not adversely affected when NAG is used in a high temperature beverage application, such as those including a pasteurization step.

### EXAMPLE 6

### Caramels

Made from scratch caramels were used as the basis for incorporation of samples. One serving is two caramels. For servings which included GLCN or NAG, the serving (2 caramels) had 0.75 g GLCN or NAG, or 0.375 g per caramel added which reflects a typical amount of GLCN or NAG delivered in dietary supplement products. Batches of 8 servings were prepared. For servings that included GLCN or NAG, 6 g GLCN or NAG was added (8 servings×0.75 g/serving=6 g).

A Better Homes & Gardens recipe was used. To make the caramels, ¼ cup non-corn oil margarine was melted over low heat in a nonstick-coated saucepan. Packed brown sugar (½ cup), ½ cup half and half (substituted for light cream), and ¼ cup light corn syrup was added and mixed well. GLCN or NAG was added to the appropriate batches, and the samples stirred well. The samples were cooked and stirred over medium-high heat to boiling. The mixture was cooked and stirred over medium heat to 248° F. until a firm-ball stage was reached. The saucepan was removed from the heat, ¼ teaspoon imitation vanilla added to the mixture, then the mixture immediately poured into a pan lined with buttered-foil and allow to cool.

Samples were tested according to the methodology and instructions in Example 1. The 12 Panelists' results are shown in Table 6.

TABLE 6
Caramel results.*

| | | | Mouthfeel/ | | |
|---|---|---|---|---|---|
| Sample | Color | Odor | texture | Taste | Comments (other than “same”) |
| Control | 0 | 0 | 0 | +0.1 | |
| NAG | 0 | −0.2 | −0.3 | −0.2 | Lighter color, smell is masked, more grainy (3), |
| | | | | | sweeter, a little better than control, didn't taste as |
| | | | | | buttery, a little aftertaste, not as sweet (3), not as |
| | | | | | chewy |
| Fungal | −1.7 | −0.3 | −0.5 | −3.5 | Darker in color (7), burnt caramel taste (2), harder |
| GLCN | | | | | than control (2), salty and solvent taste, tasted bad, |
| | | | | | bitter (7), not very pleasant, reduced caramel and |
| | | | | | butter flavor, tart, too tangy, less sweet odor, burnt |
| | | | | | odor, sour |
| Shellfish | −1.4 | −0.3 | −0.9 | −3.5 | Darker in color (7), burnt caramel taste, salty and |
| GLCN | | | | | solvent taste, tasted bad, sour (2), after taste hits |
| | | | | | hard, acidic tart, suppressed caramel & buttery |
| | | | | | flavor, almost rancid or salty, less sweet odor, bitter |
| | | | | | (7), burnt odor |

*Results are the average rounded to nearest tenth.

The results shown in Table 6 were validated; based on the control score of near “0” in all categories, the control was identified. As shown in Table 6, the taste of NAG was preferred to either GLCN sample, and NAG rated at or near control levels. Although three panelists did note that the NAG sample was more grainy and less sweet, the scores do not reflect that was perceived as highly unfavorable. These results demonstrate that NAG has an advantage over GLCN when used in a high temperature food application, as NAG does not adversely affect taste or browning after being heated as does GLCN.

### EXAMPLE 7

### Jiffy® Corn Muffin Mix

Jiffy® Corn Muffin Mix was used as the basis for incorporation of samples. Corn muffins were chosen due to their light color and mild taste. One serving is one muffin (as per the manufacturer).

Two boxes prepare 12 large muffins, a box was used to prepare 4 batches of samples (3 muffins per batch). For servings which included GLCN or NAG, the serving (1 muffin) had 0.75 g GLCN or NAG added, which reflects a typical amount of GLCN or NAG delivered in dietary supplement products. Except for the control, each batch had 2.25 g of NAG or GLCN added (0.75 g NAG or GLCN/muffin×3 muffins=2.25 g NAG or GLCN/batch). Each batch was prepared separately, so that the muffins were baked in the same places on the muffin pan. GLCN or NAG was added to the dry mix and mixed with a spoon. To this, 4 teaspoons of milk and ¼ of a beaten egg were added and the ingredients blended until slightly lumpy (each batch was mixed for the same amount of time). The mixtures were allowed to sit for three minutes, then evenly distributed into lightly-greased muffin cups. The muffins were baked at 400° F. for 18 minutes, then immediately removed from the muffin cup and stored in airtight containers.

Samples were tested according to the methodology and instructions in Example 1. The 12 Panelists' results are shown in Table 7.

TABLE 7
Jiffy ® Corn muffin results.*

| | | | Mouthfeel/ | | |
|---|---|---|---|---|---|
| Sample | Color | Odor | texture | Taste | Comments (other than “same”) |
| Control | −0.3 | −0.1 | 0 | −0.3 | Darker (2), smells sweeter, tastes sweeter, |
| | | | | | bitter, drier |
| NAG | +0.3 | −0.2 | −0.1 | −0.1 | Not good texture, slight aftertaste, lighter color |
| | | | | | (2), more sweet/less corn taste, more flour |
| | | | | | texture, less odor, better than control |
| Fungal | −2.1 | −0.3 | −0.8 | −2.4 | Darker (8), dark specs (2) crunchy, grainy, |
| GLCN | | | | | drier, bitter (4), unpleasant taste (6), burnt |
| | | | | | smell, soapy mouthfeel, less odor (3), chewier, |
| | | | | | salty/solvent taste, sour milk taste, sweeter |
| Shellfish | −1.7 | −0.4 | −0.1 | −1.0 | Darker (6), dark specs (5), alum like taste, |
| GLCN | | | | | bitter (3), less odor (2), burnt odor, “fresher”, |
| | | | | | more flour texture (2), less taste, less sweet, |
| | | | | | grainy, sour, drier |

*Results are the average rounded to nearest tenth.

The results shown in Table 7 were validated; based on the control score of near “0” in all categories, the control was identified. As shown in Table 7, the taste of NAG was preferred by the panelists in corn muffins to either GLCN sample. NAG performed at or slightly better than the control. Differences in taste were noticed by a few panelists in the crusts vs. the non-crust portion of muffins. These results demonstrate that NAG has an advantage over GLCN when used in a high temperature food application, as NAG does not appear to adversely affect taste or browning after being heated, unlike GLCN.

### EXAMPLE 8

### Hy-Vee® Fudge Brownie Mix

Hy-Vee® Fudge Brownie Mix was used as the basis for incorporation of samples. Brownies, which have a darker color, were used to mask any browning from panelists as browning could cause bias by the obvious presence of a non-control sample. According to the manufacturer, one box makes 15 brownies (1 serving is 1 brownie). To prepare the samples, the dry mix was separated into four batches, each with 3.75 servings.

For servings which included GLCN or NAG, the serving (1 brownie) had 0.75 g GLCN or NAG added, which reflects a typical amount of GLCN or NAG delivered in dietary supplement products. Except for the control GLCN or NAG was added to each batch (0.75 g NAG or GLCN/brownie×3.75 brownies=2.8 g NAG or GLCN/batch). Each batch was prepared separately, so that the brownies were baked in the same places on cookie sheet. The “Quick Brownie Cookie” recipe on the box was used. GLCN or NAG was added to the dry mix and mixed with a spoon. To each batch, 4 teaspoons cool water, 2¼ teaspoons vegetable oil and ¼ beaten egg was added. The mix was stirred with a spoon (about 40 strokes) until smooth and shiny. The mixture was dropped by teaspoonfuls onto a greased cookie sheet and baked for 10 minutes at 350° F. After baking, the brownies were immediately removed from the cookie sheet, allowed to cool, and stored in airtight containers.

Samples were tested according to the methodology and instructions in Example 1. The 12 Panelists' results are shown in Table 8.

TABLE 8
Hy-Vee ® Fudge Brownie results.*

| | | | Mouthfeel/ | | |
|---|---|---|---|---|---|
| Sample | Color | Odor | texture | Taste | Comments (other than “same”) |
| Control | 0 | 0 | −0.1 | 0 | Slightly drier, odor masked, less chewy |
| NAG | −0.6 | −0.4 | −0.6 | −0.7 | Chewy (3), exterior shiny (2), reddish color, |
| | | | | | odor masked (2), less sweet, more chocolate |
| | | | | | taste, smoother texture, taste off a little but not |
| | | | | | too bad, darker, smells salty/burnt, tastes burnt |
| Fungal | −3.1 | −2.3 | −3.6 | −4.1 | Rough texture, strong aftertaste (2), sour (2), |
| GLCN | | | | | bitter (3), tangy, burnt taste (5), burnt (8), burnt |
| | | | | | smell (3), sour odor, crispy (7), darker (6), dry, |
| | | | | | sticks to teeth |
| Shellfish | −0.4 | −0.8 | −1.3 | −2.7 | Strong aftertaste (2), smooth texture, chewy |
| GLCN | | | | | (4), sour (2), bitter (3), darker, reddish color, |
| | | | | | exterior shiny, bitter smell, rancid smell, rancid |
| | | | | | taste, crispy granules (2), less sweet, darker, |
| | | | | | masked odor, taste salty/burnt, burnt taste |

*Results are the average rounded to nearest tenth.

The results in Table 8 were validated; based on the control score of near “0” in all categories, the control was identified. As shown in Table 8, taste of NAG was preferred by the panelists more than either GLCN sample. These results demonstrate that NAG has an advantage over GLCN when used in a high temperature food application, as NAG does not appear to adversely affect taste or browning after being heated to 350° F., unlike GLCN.

### EXAMPLE 9

Fleischmann's® Country White Bread Machine Mix

Fleischmann's® Bread Machine Mix was used as the basis for incorporation of samples. Country White Mix was chosen due to its light color and mild flavor. According to the manufacturer, 1 box make 8 servings (8 slices).

One box of bread mix was used for each batch. For servings which included GLCN or NAG, the serving (1 slice) had 0.75 g GLCN or NAG added, which reflects a typical amount of GLCN or NAG delivered in dietary supplement products. Except for the control, 6 g GLCN or NAG was added to each batch (0.75 g NAG or GLCN/slice×8 slices=6 g NAG or GLCN/batch). The recipe on the box was used. GLCN or NAG was added to the dry mix and mixed with a spoon, and the mixture added to a breadmaker. To this, 8 ounces of water (75° F.-85° F.), and 1 package of yeast (provided with the bread mix) was added. The bread machine was set to medium/normal crust color. The finished bread was immediately removed, cooled on a plate, and stored in airtight containers.

Samples were tested according to the methodology and instructions in Example 1. The 10 Panelists' results are shown in Table 9.

TABLE 9
Fleischmann's ® Country White Bread Machine Mix results.*

| | | | Mouthfeel/ | | |
|---|---|---|---|---|---|
| Sample | Color | Odor | texture | Taste | Comments (other than “same”) |
| Control | 0 | 0 | −0.3 | −0.2 | Less odor, drier (2) |
| NAG | −0.1 | 0 | 0 | −0.3 | Sour, darker, darker crust, tougher, heavier texture, |
| | | | | | more porous (2), salty/briny aftertaste |
| Fungal | −0.9 | −0.3 | −1.1 | −1.0 | Doughy (4), darker (2), darker crust, darker bread, |
| GLCN | | | | | denser (3), spongier, chewier (4), earthy taste, |
| | | | | | sour, sweeter (2), bitter, moister (3), “breadier” |
| | | | | | smell, crust slightly crisp, yeasty, gummy after |
| | | | | | chewed |
| Shellfish | −0.9 | −0.2 | −0.4 | −1.1 | Darker (2), darker crust, darker bread, sour, |
| GLCN | | | | | spicy/musty odor, denser, spongier (2), earthy |
| | | | | | taste, bitter taste (2), burnt taste, vinegar taste, |
| | | | | | moister, “breadier” smell, chewier, more bread |
| | | | | | taste |

*Results are the average rounded to nearest tenth.

The results shown in Table 9 were validated; based on the control score of near “0” in all categories, the control was identified. As shown in Table 9, the taste of NAG was preferred by the panelists more than either GLCN sample, with NAG performing about as well as the control. These results demonstrate that NAG has an advantage over GLCN when used in a high temperature food application, as NAG does not appear to adversely affect taste or browning after being baked at high temperatures, unlike GLCN.

### EXAMPLE 10

### Hy-Vee® Healthy Recipe Tomato Soup

Hy-Vee® Healthy Recipe Tomato Soup was used as the basis for incorporation of samples. Tomato soup was chosen for its even texture, and “healthy” aspect as compared to other soups. According to the manufacturer, one can makes 2.5 servings.

For servings that included GLCN or NAG, the serving had 0.75 g GLCN or NAG added, which reflects a typical amount of GLCN or NAG delivered in dietary supplement products. Each batch prepared 2.5 servings, so except for the control, GLCN or NAG was added to each batch (0.75 g GLCN or NAG/serving×2.5 servings=1.875 g GLCN or NAG/batch). The recipe on the can was used. The soup can contents were poured into a microwaveable container, one can of water slowly stirred in, and GLCN or NAG added (where applicable) and the mixture stirred well. The container was covered with vented plastic wrap (turn back edge of wrap to form small opening for steam to escape), and the mixture heated in a 1000 watt Amana Radarange at high power for 2 minutes. The sample was not allowed to come to a boil.

Samples were tested according to the methodology and instructions in Example 1. The 8 Panelists' results are shown in Table 10.

TABLE 10
Hy-Vee ® Healthy Recipe Tomato Soup results.*

| | | | Mouthfeel/ | | Comments |
|---|---|---|---|---|---|
| Sample | Color | Odor | texture | Taste | (other than “same”) |
| Control | −0.2 | 0 | −0.1 | +0.2 | less tangy (2), lighter |
| | | | | | in color |
| NAG | 0 | 0 | 0 | 0 | Sweeter, less acidic with |
| | | | | | weaker aftertaste |
| Fungal | 0 | 0 | −0.1 | −0.3 | Off flavor, a little |
| GLCN | | | | | metallic, flat/sour taste |
| | | | | | but barely perceptible, |
| | | | | | more tangy |
| | | | | | w/acidic aftertaste (like |
| | | | | | black pepper had been |
| | | | | | added), milder, maybe |
| | | | | | sweeter, less tangy |
| | | | | | aftertaste |
| Shellfish | −0.2 | 0 | −0.3 | −0.6 | Slight off flavor, dirty |
| GLCN | | | | | odor, flat/sour taste but |
| | | | | | barely perceptible, lighter |
| | | | | | in color, off aftertaste, |
| | | | | | bitter, chalky in texture, |
| | | | | | sour |

*Results are the average rounded to nearest tenth.

The results shown in Table 10 were validated by identification of the control; based on the control score of near “0” in all categories, with NAG scoring as well as the control. Even though it was difficult for the panelists to ascertain differences between the samples, the NAG was favored over the GLCN samples. These results further demonstrate that taste and other factors are not adversely affected when NAG is used in a high temperature food application, such as those which include heating prior to consumption.

### EXAMPLE 11

### Bubble Gum

Canny Kits® Bubble Gum was be used as the basis for incorporation of samples. A serving of gum was estimated to be two pieces of gum.

According to the manufacturer's instructions, the mix makes over ¼ pound of gum. Assuming a piece of gum weighs at least 4 g, the mix can prepare approximately 30 pieces of gum. A gum kit was used to make two batches of gum. For servings which included GLCN or NAG, the serving (2 pieces of gum) had 0.75 g GLCN or NAG, or 0.375 g GLCN or NAG per piece of gum added, which reflects a typical amount of GLCN or NAG delivered in dietary supplement products. Except for the control batch, 5.625 g of GLCN or NAG was added (0.375 g active per piece of gum×15 pieces of gum in ½ a gum kit=5.625 g).

To make the gum, a plate was covered with waxed paper. Half of the powdered sugar provided was poured onto the waxed paper, and a well was formed in the pile of sugar to receive the melted gum and syrup. The gum-base pellets were melted by placing them in a plastic cup with the corn syrup, flavoring and GLCN or NAG (as applicable), then microwaving in a 1000 watt Amana Radarange microwave on high for 15 seconds. The heating step was repeated for two more 15 second intervals, stirring after each, until the gum base was completely melted. This melted mixture was slowly added to the powdered sugar, with constant mixing. The gum was worked into the powdered sugar until it no more sugar would absorb. When the gum was cool enough to handle, it was handled like bread dough and rolled flat, cooled, and cut into pieces for sensory testing.

Samples were tested according to the methodology and instructions in Example 1. The twelve Panelists' results are shown in Table 11.

TABLE 11
Results of Canny Kits ® Bubble Gum.*

| | | | Mouthfeel/ | | Comments |
|---|---|---|---|---|---|
| Sample | Color | Odor | texture | Taste | (other than “same”) |
| Control | | | +0.1 | −0.1 | Not as sweet, good |
| NAG | +0.1 | | −0.2 | −0.3 | Drier (2), crumblier, |
| | | | | | weaker texture, less |
| | | | | | fruity taste, waxy, |
| | | | | | not as soft, metallic |
| | | | | | aftertaste |
| Fungal | −0.3 | | −0.6 | −2.9 | Grainy, bitter (4), salty |
| GLCN | | | | | (3), bad, darker stale |
| | | | | | musky taste |
| Shellfish | −0.2 | | −0.8 | −1.8 | Grainy (2), bitter (2), |
| GLCN | | | | | stickier, salty (2), good, |
| | | | | | harder, stale musky |
| | | | | | taste, less fruity taste |

*Results are the average rounded to nearest tenth.

As shown in Table 11, the results were validated; based on the control score of near “0” in all categories, the control was identified. The taste of NAG was preferred by the panelists in gum over either GLCN, and the NAG was rated at or near control levels. These results demonstrate that NAG has an advantage over GLCN when used in a high temperature food application, as NAG does not appear to adversely affect taste after being heated, unlike GLCN.

### EXAMPLE 12

### Tea

Tea made from Lipton® instant tea mix was used as the basis for incorporation of samples. To prepare the tea, the following recipe was used: 2.69 g instant tea, 5.7 g GLCN or NAG (where applicable) then add water to one quart. Tea was prepared for sensory testing as follows:

Batch 1. Control Tea (no NAG or glucosamine)

Batch 2. Tea+glucosamine (pH 5-6) [GLCN from fungal biomass, see batch 2 Example 1]

Batch 3. Tea+NAG (pH 5-6) (N-Acetyl-D-glucosamine, Catalog # A-102-2, Ferro Pfanstiehl, Waukegan, Ill.)

Batch 4. Tea+glucosamine (pH 5-6) heated to 71° C. for 20 minutes

Batch 5. Tea+glucosamine heated (pH 5-6) to 90° C. for 20 seconds, flash cooled in ice water bath

Batch 6. Tea+glucosamine (pH 5-6) boiled on stove for 5 minutes, flash cooled in ice water bath

Batch 7. Tea+NAG (pH 5-6) heated to 71° C. for 20 minutes

Batch 8. Tea+NAG (pH 5-6) heated to 90° C. for 20 seconds, flash cooled in ice water bath

Batch 9. Tea+NAG (pH 5-6) boiled on stove for 5 minutes, flash cooled in ice water bath

The recipe prepared one quart, so each batch consisted of 32 ounces, or four 8-ounce servings. For servings that included GLCN or NAG, the serving (8 ounces) had 1.425 g GLCN or NAG, or 5.7 g GLCN or NAG per batch (1.425 g/serving×4 servings/batch=5.7 g/batch), which reflects a typical amount of GLCN or NAG delivered in dietary supplement products.

Samples were tested within one day of preparing. Samples were tested blindly against a marked control, and a blind control was included. Panelists were asked to compare each sample to the control and comment using the instructions and scale described in Example 1. The six panelists' results are shown in Table 12.

TABLE 12
Results of Tea.*

| | Actual | Initial | pH after | 30 sec | | | Sensory Panel |
|---|---|---|---|---|---|---|---|
| Sample | Temp ° C. | pH | heating | Temp ° C. | Mouthfeel | Taste | Observations |
| Control | Not | 5.47 | N/A | N/A | 0.7 | 3.0 | Normal, like |
| | heated | | | | | | Lipton, slightly |
| | | | | | | | dry |
| GAP | Not | 5.09 | N/A | N/A | −0.7 | −3.2 | Salty, syrupy |
| | heated | | | | | | texture, sweeter, |
| | | | | | | | bitter |
| GAP | 71 | 5.09 | 5.13 | 69 | −0.5 | −3.7 | Sweet aftertaste, |
| 71° C. | | | | | | | bitter, salty |
| Tea + GAP | 90 | 5.09 | 5.00 | 78 | −0.5 | −4.2 | Salty (2), sweet, |
| 90° C. | | | | | | | sour (2), bitter, a |
| | | | | | | | little darker |
| GAP | 94 | 5.09 | 4.87 | 74 | −0.7 | −2.2 | Sweeter (2), |
| boiled | | | | | | | bitter, astringent, |
| | | | | | | | a little darker |
| NAG | Not | 5.46 | N/A | N/A | 0.7 | 2.0 | Like control, |
| | heated | | | | | | sweet (2), bitter |
| Tea + NAG | 71 | 5.46 | 5.40 | 67 | −0.2 | 1.3 | sweet |
| 71° C. | | | | | | | |
| NAG | 90 | 5.46 | 5.31 | 81 | −0.2 | 1.2 | Bitter, tangy |
| 90° C. | | | | | | | |
| NAG | 96 | 5.46 | 5.22 | 74 | −0.3 | 0.2 | Very bitter, |
| boiled | | | | | | | caramel like but |
| | | | | | | | not sweet, darker |

*Values shown are the average rounded to nearest tenth.

As shown in Table 12, NAG was preferred over GLCN in tea, which was more neutral tasting than the lemonade. The pH of the tea shows that the pH slightly decreased after heating in most cases (with both GAP and NAG).

### EXAMPLE 13

### Comparison of GLCN-HCl and GLCN-Sulfate

As described in the above, examples, use of glucosamine-hydrochloride in food and beverages heated to high temperatures, often resulted in a sour or bitter aftertaste. To determine if similar results would be obtained when glucosamine-sulfate was used, the methods described in Example 2 (sugar cookies) were repeated using a GLCN-sulfate sample in addition to a GLCN HCl sample.

GLCN-sulfate (D-Glucosamine Sulfate 2KCl) was obtained from Anhui Technology Import & Export Co., Ltd. (Hefei, P.R. China, Batch # 2002KO925, manufacturing date: Sep. 24, 2002). Cookies were prepared as described in Example 2.

Samples were tested according to the methodology and instructions in Example 1. The 12 Panelists' results are shown in Table 13.

TABLE 13
Effect of GLCN-Sulfate versus GLCN-HCl.*

| | | | Mouthfeel/ | | |
|---|---|---|---|---|---|
| Sample | Color | Odor | texture | Taste | Comments (other than “same”) |
| Control | −0.1 | −0.1 | −0.2 | −0.2 | |
| NAG | −0.2 | −0.2 | −0.8 | −0.2 | Darker(2), more vanilla odor, less odor, less sweet, |
| | | | | | sweeter(3), crispier or crunchier(5), less buttery |
| | | | | | taste, salty taste, chewy |
| Fungal | −2.5 | −1.8 | −1.1 | −3.1 | burnt(2), darker(7), doughy(2), less sweet odor, |
| GLCN | | | | | softer(2), burnt taste, bitter taste(2), sour taste(2), |
| | | | | | black specs, salty taste, burnt sugar taste (2), |
| | | | | | molasses taste, less sweet taste, tangy taste, bitter |
| | | | | | odor, pungent dirty odor, chewy |
| GLCN | −1.6 | −0.8 | −1.0 | −2.0 | Darker(5), black specs(3), doughy, less odor, |
| sulfate | | | | | softer(5), “melts” in mouth, cinnamon “hot” |
| | | | | | aftertaste, sour taste, salty taste, bitter taste, tangy |
| | | | | | taste, bitter odor, sweeter taste, chewy, less buttery |
| | | | | | odor, less buttery taste |

*Results are the average rounded to nearest tenth.

The results shown in Table 13 are validated by identification of the control; based on the control score of near “0” in all categories. NAG was liked better by the panelists in sugar cookies than either GLCN sample, and it scored almost the same as the control. Only the GLCN samples showed significant darkening, indicating adverse interactions between the food components and GLCN during heating. These results demonstrate that NAG has an advantage over GLCN (HCl and sulfate) when used in a high temperature food application, as NAG does not appear to adversely affect taste or browning after being baked at high temperatures, unlike GLCN. In addition, it appears that both forms of GLCN (HCl and sulfate), have undesirable properties when heated.

### EXAMPLE 14

### Simulation of Stomach Conditions

As described in the above examples, NAG does not impart off flavors and colors to the extent observed by glucosamine, when used as a minor (dietary supplement) ingredient in high temperature applications. To demonstrate that NAG is biologically available once in the stomach, the following methods were used.

To test the availability of NAG in the stomach, a simulated stomach environment was constructed, and a known concentration of NAG was added to simulated gastric fluid (SGF) and maintained at conditions simulating the stomach. If NAG is degraded or derivatized in the stomach, the NAG concentration will decrease. If the concentration of NAG remains the same, it is likely that the NAG is available to the body.

SGF was prepared based on a 1995 United States Pharmacopoeia (USP) monograph. An artificial stomach environment was simulated according to generally accepted practice (see Consumers Union website, 101 Truman Ave., Yonkers, N.Y. 10703-1057). To prepare simulated gastric fluid, 2.0 g of NaCl and 3.2 g of pepsin were dissolved in 7.0 mL of hydrochloric acid (concentrated) and sufficient water to make a 1000 mL solution. This solution had a pH of about 1.2.

NAG samples were prepared as follows. A typical amount of GLCN or NAG delivered in dietary supplement products is 0.5 -1.5 g/serving, and is often taken in multiple 0.5 g or 0.75 g doses, or as a single dose. NAG (1 g) was placed in the simulated stomach (SGF solution) to be at an optimum concentration for the instrument, and to reflect a reasonable ingestion of NAG as a supplement.

To simulate the stomach, experiments were conducted at 37° C., which is normal human body temperature, in a water bath/shaker, to gently agitate the samples. Beakers sealed with parafilm were used to restrict loss of sample.

Before introduction of samples into the SGF, a sample of the SGF was analyzed as a blank, to determine the baseline. Upon addition of the NAG to the SGF, a sample was immediately analyzed to determine the concentration of NAG at time zero. After introduction of NAG into the SGF, samples were pulled and analyzed at 5 minutes, 15 minutes, and 60 minutes to provide a profile of NAG concentration in the simulated stomach, and analyzed for the concentration of NAG.

After subjecting the NAG to the SFG as described above, there was no significant loss of NAG with respect to the accuracy of the method. Therefore, it is likely that the NAG is available to the body after ingestion, since it does not appear that NAG is significantly chemically altered (such as degraded or derivatized) following ingestion.

### EXAMPLE 15

### Determination of the Amount of NAG Present following Heating

To demonstrate that NAG remains following heating or pasteurization of food products and beverages, the following methods were used. Food samples were homogenized prior to analysis. Where possible, samples were frozen or dried and ground. Baked goods were crumbled and mixed to produce a homogeneous material. Suitable blanks (samples with no added NAG) of each food type were analyzed to assess interferences. Mass changes between unheated ingredient mixtures and baked final products were tracked to permit accurate recovery calculations.

Acid Extraction Method

A food or beverage sample as described in the preceding examples containing 5 to 20 mg of N-acetylglucosamine (NAG) was dispersed in 25 g of 0.1 N HCl in a 50-mL polypropylene centrifuge tube and capped tightly. The sample was mixed for 30 seconds using a vortex mixer, then placed in a water bath at 37° C. The sample was removed from the water bath at 15-minute intervals and mixed for 30 seconds on a vortex mixer and then returned to the water bath. This cycle was repeated until the sample had been in the water bath for one hour. After heating, the sample was mixed for 30 seconds on a vortex mixer, then centrifuged for 10 minutes to separate the liquid and solid phases. Fats, oils or lipids in the sample formed a third layer at the top of the tube. The aqueous portion of the sample was filtered through a 0.2μ filter into an HPLC vial, then capped.

NAG recovery was determined using high performance liquid chromatography (HPLC) using a combination of refractive index and UV (195 nm) detection. The system included a SIL-10AXL autosampler, SCL-10AVP controller, LC-10AT pump, CTO-6A column oven, SPD-M10AVP diode-array detector, and a RID-6A refractive index detector, all from Shimadzu Scientific Instruments, Inc. (Columbia, Md.). The column was a MetaCarb H Plus, 300×7.8 mm, from Varian, Inc. (Torrence, Calif.).

The eluent, 0.01N sulfuric acid in water, flow rate was 0.4 mL/min. The column was maintained at 70° C. A 10 μL injection volume was used. NAG eluted at 23.9 minutes and was well resolved from other species in the samples. Multiple standards confirmed good linearity over the concentration range of interest. The LW spectrum from 190 to 350 nm indicated no measurable co-eluting peaks, and the retention time and ratio of responses between the detectors confirmed the identity of NAG.

As shown in Table 14, the amount of NAG recovered following heating ranged from about 77%-100%.

AOAC Method

One method used to determine the amount of NAG in processed food samples was adapted from “Glucose, Fructose, Sucrose, and Maltose in Presweetened Cereals: Liquid Chromatography Method”, AOAC Method 982.14, 15^{th} Ed. (1990), pp. 789-790 (herein incorporated by reference). Specifically, section C of the method was adapted to extract NAG from dry-mixed and baked samples.

The sample was dried (if needed) then ground to render it homogeneous. Approximately five grams of sample were mixed with 100 mL of a 1:1 water:ethanol solution. The samples were heated for 30 minutes at 80-85° C. After heating, ethanol was added to replace evaporated solvent. The supernatant and solids were separated by centrifugation followed by filtration. The supernatant was analyzed by HPLC to determine the NAG content using standard methods, including a BioRad HPX-87H column heated to 60° C., 0.01 N H₂SO₄ mobile phase at 0.6 mL/minute, and a refractive index detector.

Suitable blanks (samples with no added NAG) of each food type were analyzed to assess interferences. Mass changes between dry mixes and baked final product were tracked to permit accurate recovery calculations.

Using the AOAC method NAG was recovered as follows: For bread, the dry bread mix and baked product yielded 93% and 80% recoveries of NAG, respectively. For cookies, the dry cookie mix and baked recoveries of NAG were 78% and 68%, respectively. Therefore, the majority of NAG is unchanged when exposed to a high temperature, and is available to a subject upon ingestion of the heated food or beverage supplemented with NAG. These results compare favorably to the acid-extraction method of recovery of 100% and 77%, respectively.

### EXAMPLE 16

### Determination of the Amount of Glucosamine Present following Heating

To determine the amount of glucosamine that remains following heating or pasteurization of food products and beverages, the following methods were used. Food samples were homogenized prior to analysis. Where possible, samples were frozen and ground. Baked goods were crumbled and mixed to produce a homogeneous material.

A food or beverage sample as described in the preceding examples containing 5 to 20 mg of GLCN was dispersed in 25 g of 1.0 N HCl in a 50-mL polypropylene centrifuge tube and capped tightly. The sample was mixed for 30 seconds using a vortex mixer, then placed in a water bath at 37° C. The sample was removed from the water bath at 15-minute intervals, mixed for 30 seconds on a vortex mixer, then returned to the water bath. This cycle was repeated until the sample had been in the water bath for one hour.

After heating, the sample was mixed for 30 seconds on a vortex mixer, then centrifuged for 10 minutes to separate the liquid and solid phases. Fats, oils or lipids in the sample formed a third layer at the top of the tube. A 1-g aliquot of the aqueous sample portion was diluted 100-fold with deionized water, then transferred to an autosampler vial with filter cap.

The free glucosamine in prepared samples was determined using high performance anion-exchange chromatography with pulsed amperometric detection (HPAEC-PAD). The system included an EG40 eluent generator, GP50 gradient pump, AS40 autosampler, LC25 column oven, and ED40 electrochemical detector, all produced by Dionex Corporation (Sunnyvale, Calif.).

The method was adapted from Dionex Corporation Technical Note 40. A Dionex CarboPac PA-20 column was used in place of the PA-10 described in the Technical Note. The eluent was 8 mM KOH at 0.5 mL/min. The column and detector were maintained at 30° C. The injection volume was 10 μL. The standard was glucosamine hydrochloride at 10.8 mg/L. Fermentation broth samples were diluted five-fold with deionized water, ASTM Type II, and filtered through 0.2μ vial filters in the autosampler. Multiple standards were analyzed before and after each sample set. The results are shown in Table 14.

TABLE 14
Percent of NAG and GLCN recovery following heating.

| Sample | Temp° F. | Temp° C. | Time | | NAG % | GlcNHCl % |
|---|---|---|---|---|---|---|
| Chocolate morsels (Example 4) | | 52 | 1 | min | 97 | 97 |
| Rice Krispies ® (Example 1) | 165 | 74 | <10 | min | | 106 |
| Bread mix (Example 9) | 250 | 121 | 50 | min | 100 | 46 |
| Sugar cookies (Example 2) | 375 | 191 | 10 | min | 77 | 50 |
| Corn muffin mix (Example 7) | 400 | 204 | 18 | min | 77 | 13 |
| Tea (Example 12) | | Room temp | | | 98 | 96 |
| Tea (Example 12) | | 90 | 20 | sec | 98 | 94 |
| Tea (Example 12) | | 71 | 20 | min | 98 | 100 |
| Tea (Example 12) | | boil | 5 | min | 96 | 93 |

As shown in Table 14, there was some degradation of NAG and GLCN when the food products were baked or boiled. NAG was lesser affected by heating than was GLCN. The amount of NAG recovered ranged from about 77%-100%, while the amount of recovery for GLCN varied more widely from about 13%-100%, for example about 46%-100%.

In view of the many possible embodiments to which the principles of this disclosure may be applied, it should be recognized that the illustrated embodiments are only particular examples of the disclosure and should not be taken as a limitation on the scope of the disclosure. Rather, the scope of the disclosure is in accord with the following claims. We therefore claim all that comes within the scope and spirit of these claims.

We claim:

1. A beverage comprising:
a heat pasteurized NAG beverage, wherein the beverage comprises at least about 0.01 g NAG per serving.

2. The beverage of claim 1, wherein the heat pasteurized NAG beverage comprises at least about 250 mg to about 1500 mg NAG per serving.

3. The beverage of claim 1, wherein the heat pasteurized NAG beverage is at a temperature of at least about 160° F.

4. The beverage of claim 3, wherein the heat pasteurized NAG beverage is at a temperature of at least about 180° F.

5. The beverage of claim 1, wherein the heat pasteurized NAG beverage is at a temperature of about 161° F. to about 300° F.

6. A method of preparing a beverage, comprising
providing a beverage;
adding at least about 0.01 g NAG per serving to the beverage to form a NAG beverage; and
heat pasteurizing the NAG beverage at a temperature of least about 160° F.

7. The method of claim 6, wherein the NAG beverage is heat-pasteurized at a temperature of at least about 200° F.

8. The method of claim 6, wherein an amount of NAG present in the NAG beverage is about 250 mg to about 1500 mg NAG per serving.

9. The method of claim 6, wherein the NAG is derived from fungal biomass containing chitin.

10. A food product comprising:
a NAG food product comprising at least about 0.01 g NAG per serving, wherein the NAG food product is at a temperature of at least about 160° F.; and
an absence of shellfish proteins.

11. The food product of claim 10, wherein the NAG food product is at a temperature of at least about 200° F.

12. The food product of claim 10, wherein the food product is a flour- or grain-based product.

13. The food product of claim 1 0, wherein an amount of NAG present in the NAG food product is about 250 mg to about 1500 mg NAG per serving.

14. A method of preparing a food product, comprising
providing a food product;
adding a first amount of NAG derived from fungal biomass containing chitin to the food product to form a NAG food product, wherein the NAG food product comprises at least about 0.01 g NAG per serving; and
heating the NAG food product to a temperature of at least about 160° F.

15. The method of claim 14, wherein the heating comprises baking, broiling, or boiling the NAG food product.

16. The method of claim 14, wherein the first amount of NAG present in the NAG food product is about 250 mg to about 1500 mg per serving.

17. The method of claim 14 wherein the NAG food product is heated to a temperature of at least about 200° F.

18. The method of claim 6, wherein at least about 0.007 g NAG per serving remains in the NAG beverage after heat pasteurizing.

19. The method of claim 14, wherein a second amount of NAG present in the NAG food product after heating the NAG food product is at least about 70% of the first amount of NAG present in the NAG food product before heating the NAG food product.

20. The beverage of claim 1 or the food product of claim 10, wherein NAG comprises at least 1% of a sweetener in the beverage or food product.

21. A non-acidified food product, comprising:
a food product comprising at least about 0.01 g NAG per serving; thereby generating a NAG food product, wherein the NAG food product is at a temperature of at least about 160° F.
